(19) United States
(12) Patent Application Publication
(10) Pub. No.: US 2024/0390258 A1
(43) Pub. Date: Nov. 28, 2024

(54) FOAMING COMPOSITION

(71) Applicant: L'OREAL, Paris (FR)

(72) Inventors: Chunyue LIU, Shanghai (CN); Yong WANG, Shanghai (CN); Nariyoshi YOSHIOKA, Shanghai (CN)

(73) Assignee: L'OREAL, Paris (FR)

(21) Appl. No.: 18/693,848

(22) PCT Filed: Sep. 27, 2021
(86) PCT No.: PCT/CN2021/120908

§ 371 Date: Mar. 20, 2024

(51) Int. Cl. A61K 8/87; A61K 8/04; A61Q 1/14; A61Q 19/10

(52) U.S. Cl. CPC A61K 8/87; A61K 8/046; A61Q 1/14; A61Q 19/10; A61K 2800/52; A61K 2800/5422; A61K 2800/5428; A61K 2800/592; A61K 2800/596

(57) ABSTRACT

Disclosed is a foaming composition, comprising: (a) at least one oil. (b) at least one polymeric interface stabilizer of improving rheology of the composition and boosting and stabilizing the foams formed therein, and (c) a surfactant system, comprising, substantially consisting of, or even consisting of at least one alkyl sulfosuccinate, at least one amphoteric surfactant, and optionally, at least one nonionic surfactant.

### TECHNICAL FIELD

The present invention relates to a foaming composition. More particularly, the present invention relates to a foaming composition for cleansing and/or removing makeup from keratin materials.

### BACKGROUND ART

Cleansing the skin and removing makeups on the skin are very important for caring for the skin. In particular, it is very important to consumers with makeup products to have an efficient makeup remover. It must be as efficient as possible because greasy residues, such as dirt, excess sebum, and the remnants of cosmetic products used daily, and make-up products, in particular waterproof products, accumulate in the skin folds, and can block the pores of the skin and result in the appearance of spots.

So far, there are several types of makeup removing or cleansing products, for example, rinsable cleansing anhydrous oils and gels, solid makeup removers, biphasic removers, remover lotion or cream and etc.

Among them, foaming cleansing products are favorable, which have a cleansing action by virtue of the surfactants, and suspend the fatty residues and the pigments of, for example, make-up products. A good foaming property, rinsability, and skin-care property, for example, leaving a good feeling on the skin after rinsing off, such as skin mildness and a moisturizing sensation, are very important for cosmetic foaming cleanser products.

Conventionally, the products for cleansing the skin and removing makeups on the skin contain high content of oils, or even are pure oil products. These products often have poor foaming properties, and thus it is a challenge for the artisans to achieve desirable foaming properties in the products containing high content of oils.

From the consumer's perspective, the amount of the foam directly relates to the perceived cleaning efficiency of the composition. Generally speaking, the larger the volume of foam produced and the more stable the foam is, the more efficient cleaning action of the composition is the perceived.

Nowadays it has become more and more important that cleansing compositions provide effective cleansing of the keratin materials, perceived and desirable foams, and no irritation to eyes and the keratin materials, rendering the keratin materials fresh and clean, even for the products containing high content of oils.

Thus, there is a need for formulating such a composition for cleansing and/or removing makeup from keratin materials.

### SUMMARY OF THE INVENTION

According to the first aspect, the present invention relates to a foaming composition, comprising: (a) at least one oil, (b) at least one interface stabilizer of improving rheology of the composition and boosting and stabilizing the foams formed therein, and (c) a surfactant system, comprising, substantially consisting of, or consisting of at least one alkyl sulfosuccinate, at least one amphoteric surfactant, and optionally, at least one nonionic surfactant.

Further, the interface stabilizer according to the present invention is a polyurethane polymer. Preferably, the polyurethane polymer is formed by the reaction of prepolymer (i) with a coreactant (ii), and optionally is terminated by an end-capping agent (iii), wherein the prepolymer (i) is formed by the reaction of polyhydroxyl compounds, polyisocyanates, and optionally low molecular weight diols.

By means of the specific interface stabilizer, the composition according to the present invention achieves not only improved rheology, thickening efficiency, clarity, and non-tacky feel, but also perceived foams with desirable volume, density and stability.

For the surfactant system, the alkyl sulfosuccinate may be selected from mono- or di-alkyl sulfosuccinates in which the alkyl radicals have 4 to 24 carbon atoms and the counterion to the sulfonic acid group is selected from alkali metal cations and ammonium ions; the amphoteric surfactant may be selected from optionally quaternized secondary or tertiary aliphatic amine derivatives, preferably, selected from (C₈-C₂₀) alkylbetaines, (C₈-C₂₀) alkylamido (C₁-C₆) alkylbetaines, and mixtures thereof; and the non-ionic surfactant may be alkyl poly glucoside having a C₆-C₃₄ alkyl group and a moiety derived from a reducing saccharide containing from 5 to 6 carbon atoms, preferably a glucose unit.

The at least one oil is selected from oils of plant or animal origins, ester oils, ether oils, silicone oils, hydrocarbon oils, and mixtures thereof, wherein the at least one oil is present in an amount of higher than about 10 wt. %, and even higher than about 40 wt. %, relative to the total weight of the composition; provided that when the silicone oils and/or hydrocarbon oils are present, the amount of each of the silicone oil and hydrocarbon oil ranges from about 0 wt. % to about 10 wt. %, relative to the total weight of the composition.

As an example, the weight ratio of the interface stabilizer to the at least one oil ranges from about 1:500 to about 1:1, and preferably from about 1:200 to about 1:10.

According to the second aspect, the present invention relates to a combination of improving foam properties, even for high-oil products, comprising, substantially consisting of, or even consisting of a surfactant system and at least one interface stabilizer for improving rheology of the composition and boosting and stabilizing the foams formed therein, wherein the “high-oil product” means that the oil is present in an amount of higher than 10 wt. %, and even higher than 40 wt. %, relative to the total weight of the product.

Those skilled in the art understand that, for cleansing products, the foam properties are heavily impact by the oil content therein, and a high-oil product normally has a very poor foaming property. By means of the combination according to the present invention, the high-oil products can obtain good to excellent foaming properties even at a very high oil content, e.g. an oil content of higher than 10 wt. %, and even higher than 40 wt. %, relative to the total weight of the product.

As an example, the foaming compositions according to the present invention are in the form of emulsion, lotion, balm or cream.

According to the third aspect, the present invention relates to use of a polyurethane polymer, especially that is formed by the reaction of prepolymer (i) with a coreactant (ii), and optionally is terminated by an end-capping agent (iii), wherein the prepolymer (i) is formed by the reaction of polyhydroxyl compounds, polyisocyanates, and optionally low molecular weight diols, for improving rheology of an emulsified system and boosting and stabilizing the foams formed therein.

Other subjects and characteristics, aspects and advantages of the present invention will emerge even more clearly on reading the detailed description and the examples that follow.

### DETAILED DESCRIPTION OF THE INVENTION

In that which follows and unless otherwise indicated, the limits of a range of values are included within this range, in particular in the expressions “of between” and “ranging from . . . to . . . ”.

The articles “a” and “an”, as used herein, mean one or more when applied to any feature in embodiments of the present invention described in the specification and claims. The use of “a” and “an” does not limit the meaning to a single feature unless such a limit is specifically stated. Moreover, the expression “at least one” used in the present description is equivalent to the expression “one or more”.

Throughout the present application, the expression “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”). In the case of “consisting essentially of,” any additional compositions, materials, and/or components that materially affect the basic and novel characteristics are excluded from such an embodiment, but any compositions, materials and/or components that do not materially affect the basic and novel characteristics can be included in the embodiment.

By “keratin materials” we intend to mean human keratin materials and more specifically skin and scalp, and more particularly the facial skin.

### Surfactant System

The composition according to the present invention comprises a surfactant system, which comprises, substantially consisting of, or even consisting of at least one alkyl sulfosuccinate as the anionic surfactant and at least one amphoteric surfactant, and optionally at least one nonionic surfactant. The total amount of the surfactants comprised in the surfactant system ranges from about 0.5 to about 30 wt. %, preferably from about 1 to about 20 wt. %, or from about 5 to about 15 wt. %, relative to the total weight of the composition.

Useful but non-limiting examples of surfactants that may be used in the surfactant system are provided below.

### Alkyl Sulfosuccinate

The composition according to the present invention comprises at least one anionic surfactant chosen from alkyl sulfosuccinates, including mono-alkyl sulfosuccinates and di-alkyl sulfosuccinates.

Preferably, the alkyl sulfosuccinates present in the composition according to the present invention are selected from mono- or di-alkyl sulfosuccinates in which the alkyl radicals have 4 to 24 carbon atoms, preferably 6 to 18 carbon atoms, particularly preferably 6 to 14 carbon atoms. Different or identical alkyl radicals can be present in one molecule of di-alkyl sulfosuccinate, with identical being preferred. The alkyl radicals can be linear, branched or cyclic, saturated or unsaturated, and substituted or unsubstituted.

The sulfosuccinates may be chosen from alkali metal salts such as the sodium or potassium salt and preferably the sodium salt, ammonium salts, amine salts and in particular amino alcohol salts or alkaline-earth metal salts such as the magnesium salts.

According to a particular embodiment of the invention, the sulfosuccinates are chosen from the salts of alkali metals and even more particularly the sodium salt, including disodium salts for mono-alkyl sulfosuccinates and sodium salts for di-alkyl sulfosuccinates.

Preferably, the alkyl sulfosuccinate is selected from mono- or di-alkyl sulfosuccinates in which the alkyl radicals have 6 to 14 carbon atoms and the counterion to the sulfonic acid group is selected from alkali metal cations and ammonium ions.

Non-limiting examples of the di-alkyl sulfosuccinates are diethylhexyl sodium sulfosuccinate, dinonyl sodium sulfosuccinate, diisononyl sodium sulfosuccinate, dioctyl sodium sulfosuccinate, diheptyl sodium sulfosuccinate, dihexyl sodium sulfosuccinate, dicapryl sodium sulfosuccinate, didecyl sodium sulfosuccinate, diundecyl sodium sulfosuccinate, dilauryl sodium sulfosuccinate, dicocoyl sodium sulfosuccinate, ditridecyl sodium sulfosuccinate, dipropylheptyl sodium sulfosuccinate, dicyclohexyl sodium sulfosuccinate, ammonium diethylhexyl sulfosuccinate, ammonium dinonyl sulfosuccinate, ammonium diisononyl sulfosuccinate, ammonium dioctyl sulfosuccinate, ammonium diheptyl sulfosuccinate, ammonium dihexyl sulfosuccinate, ammonium dicapryl sulfosuccinate, ammonium didecyl sulfosuccinate, ammonium diundecyl sulfosuccinate, ammonium dilauryl sulfosuccinate, ammonium dicocoyl sulfosuccinate, ammonium ditridecyl sulfosuccinate, ammonium dipropylheptyl sulfosuccinate, ammonium dicyclohexyl sulfosuccinate, diethylhexyl potassium sulfosuccinate, dinonyl potassium sulfosuccinate, diisononyl potassium sulfosuccinate, dioctyl potassium sulfosuccinate, diheptyl potassium sulfosuccinate, dihexyl potassium sulfosuccinate, dicapryl potassium sulfosuccinate, didecyl potassium sulfosuccinate, diundecyl potassium sulfosuccinate, dilauryl potassium sulfosuccinate, dicocoyl potassium sulfosuccinate, ditridecyl potassium sulfosuccinate, dipropylheptyl potassium sulfosuccinate, dicyclohexyl potassium sulfosuccinate, with diethylhexyl sodium sulfosuccinate being very particularly preferred.

Non-limiting examples of the mono-alkyl sulfosuccinates are diammonium lauryl sulfosuccinate, disodium cetearyl sulfosuccinate, disodium cetyl sulfosuccinate, disodium coco-sulfosuccinate, disodium isodecyl sulfosuccinate, disodium isostearyl sulfosuccinate, disodium lauryl sulfosuccinate, disodium oleyl sulfosuccinate, disodium stearyl sulfosuccinate, disodium tridecyl sulfosuccinate, with disodium lauryl sulfosuccinate being very particularly preferred.

Advantageously, the alkyl sulfosuccinate(s) is present in an amount ranging from about 0.5 wt. % to about 15 wt. %, preferably from about 1 wt. % to about 10 wt. %, or from about 2 wt. % to about 8 wt. %, relative to the total weight of the composition.

### Amphoteric Surfactant

The composition according to the present invention comprises at least one amphoteric surfactant chosen from optionally quaternized secondary or tertiary aliphatic amine derivatives.

The amphoteric surfactant chosen from optionally quaternized secondary or tertiary aliphatic amine derivatives contains at least one anionic group, for instance a carboxylate, sulfonate, sulfate, phosphate or phosphonate group, in which the aliphatic group or at least one of the aliphatic groups is a linear or branched chain comprising from 8 to 22 carbon atoms.

Mention may be made in particular of (C₈-C₂₀) alkylbetaines, sulfobetaines, (C₈-C₂₀ alkyl) amido (C₂-C₈ alkyl) betaines and (C₈-C₂₀ alkyl) amido (C₂-C₈ alkyl) sulfobetaines.

Among the (C₈-C₂₀) alkylbetaines, mentions may be made of behenylbetaine, cetyl betaine, cocoylbetaine, decylbetaine. From alkylbetaines, cocoylbetaine is preferred, for example the products sold by the company Rhodia under the tradename Mirataine® BB/FLA.

Among the optionally quaternized secondary or tertiary aliphatic amine derivatives that may be used, mention may also be made of the compounds of formulas (II) and (III) below:

Rₐ—CON(Z)CH₂₋(CH₂)ₘ—N⁺(R_{b})(R_{c})(CH₂COO⁻) (II)

in which:
Rₐ represents a C₁₀-C₃₀ alkyl or alkenyl group derived from an acid Rₐ—COOH preferably present in hydrolysed coconut oil, a heptyl group, a nonyl group or an undecyl group,
R_{b} represents a β-hydroxyethyl group,
R_{c} represents a carboxymethyl group;
m is equal to 0, 1 or 2,
Z represents a hydrogen atom or a hydroxyethyl or carboxymethyl group;

R_{a′}—CON(Z)CH₂—(CH₂)_{m′}—N(B)(B′) (III)

in which:
B represents —CH₂CH₂OX′, with X′ representing —CH₂—COOH, CH₂—COOZ′, —CH₂CH₂—COOH, —CH₂CH₂—COOZ′, or a hydrogen atom,
B′ represents —(CH₂)_{z}—Y′, with z=1 or 2, and Y′ representing —COOH, —COOZ′, —CH₂—CHOH—SO₃H or —CH₂—CHOH—SO₃Z′, m′ is equal to 0, 1 or 2,
Z represents a hydrogen atom or a hydroxyethyl or carboxymethyl group,
Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris (hydroxymethyl) aminomethane,
Rₐ′ represents a C₁₀-C₃₀ alkyl or alkenyl group of an acid Rₐ COOH preferably present in hydrolysed linseed oil or coconut oil, an alkyl group, in particular a C₁₇ alkyl group, and its iso form, or an unsaturated C₁₇ group.

The compounds corresponding to formula (II) are preferred.

Among the compounds corresponding to formula (II), mentions may be made of cocamidopropyl betaine, for example the product sold under the tradename Dehyton PK 45 by Cognis (BASF).

Use may also be made of the compounds of formula (IV):
Rₐ “—NH—CH(Y”)—(CH₂)ₙ—C(O)—NH—(CH₂) n′—N(Rₐ) (Re) (IV) in which:
Rₐ″ represents a C₁₀-C₃₀ alkyl or alkenyl group of an acid R_{a″}—C(O) OH preferably present in hydrolysed linseed oil or coconut oil;
Y″ represents the group —C(O)OH, —C(O) OZ″, —CH₂—CH(OH)—SO₃H or the group —CH₂—CH(OH)—SO₃—Z″, with Z″ representing a cationic counterion resulting from an alkali metal or alkaline-earth metal, such as sodium, an ammonium ion or an ion resulting from an organic amine;
R_{d} and Rₑ represent, independently of each other, a C₁-C₄ alkyl or hydroxyalkyl radical; and
n and n′ denote, independently of each other, an integer ranging from 1 to 3.

Among the compounds corresponding to formula (IV), mention may in particular be made of the compound classified in the CTFA dictionary under the name sodium diethylaminopropyl cocoaspartamide, such as the one sold by the company Chimex under the name Chimexane HB.

Preferably, the amphoteric surfactants are selected from (C₈-C₂₀) alkylbetaines, (C₈-C₂₀) alkylamido (C₁-C₆) alkylbetaines, and mixtures thereof.

More preferably, the amphoteric surfactant is selected from cocamidopropyl betaine, cocoylbetaine, or a mixture thereof.

Advantageously, the amphoteric surfactant is present in an amount ranging from about 0.5 wt. % to about 20 wt. %, preferably from about 1 wt. % to about 10 wt. %, or from about 2 wt. % to about 5 wt. %, relative to the total weight of the composition.

### Non-Ionic Surfactant

The composition according to the present invention optionally comprises at least one non-ionic surfactant selected from alkyl poly glucoside, alkyl glycoside, acyl glucamide and mixtures thereof, and preferably selected from alkyl poly glucoside.

Further, the alkyl poly glucoside preferably has the following formula (I):

R—O-G_{x′} (I)
wherein
R is a C₄-C₄₀ alkyl group,
G is a moiety derived from a reducing saccharide containing from 5 to 6 carbon atoms, preferably a glucose unit, and
x′ represents the average degree of polymerisation of the alkyl poly glucoside.

Preferably, R is a C₆-C₃₄ alkyl group.

For a particular alkyl polysaccharide molecule, x′ can only assume integral values. In any physical sample of alkyl poly glucosides, there will generally be molecules having different values of x′. The physical sample can be characterized by the average value of x′, which can assume non-integral values. In the specification, the values of x′ are to be understood to be average values.

The polysaccharide hydrophilic portion of the alkyl poly glucoside contains from about 1 to about 10, preferably from 1.4 to 3, saccharide units on the average. The saccharide unit may be galactoside, glucoside, lactoside, fructoside, glucosyl, fructosyl, lactosyl, and/or galactosyl units, and preferably glucoside unit. Mixtures of these saccharide moieties may be used in the alkyl poly saccharide. Glucoside is the preferred saccharide moiety, other saccharide moieties will act similarly, but because glucoside is the preferred saccharide moiety, the remaining disclosure will focus on the alkyl poly glucoside.

The hydrophobic group on the alkyl poly glucoside is an alkyl group, branched or unbranched, unsaturated or saturated, containing from about 4 to about 40 carbon atoms on average. Preferably the alkyl group is primarily a straight chain saturated C₆ to C₃₄ alkyl group.

Useful alkyl poly glucosides of the present invention are also disclosed in U.S. Pat. No. 4,565,647, Llenado, issued Jan. 21, 1986, having a hydrophobic group containing from about 6 to about 30 carbon atoms, preferably from about 10 to about 16 carbon atoms and a polysaccharide, e.g., a poly glycoside, hydrophilic group containing from about 1.3 to about 10, preferably from about 1.3 to about 3, most preferably from about 1.3 to about 2.7 saccharide units. Any reducing saccharide containing 5 or 6 carbon atoms can be used, e.g., glucose, galactose and galactosyl moieties can be substituted for the glucosyl moieties (optionally the hydrophobic group is attached at the 2-, 3, 4-, etc positions thus giving a glucose or galatose as opposed to a glucoside or galactoside). The intersaccharide bonds can be, e.g., between the one position of the additional saccharide units and the 2-, 3-, 4-, and/or 6-positions on the preceding saccharide units.

Examples of alkyl poly glucoside include but are not limited to: caprylyl/capryl glucoside, decyl glucoside, lauryl glucoside, octyl glucoside, sodium lauryl glucose carboxylate (and) lauryl glucoside, and coco glucoside.

Typically, the non-ionic surfactant is selected from the group consisting of caprylyl/capryl glucoside, coco glucoside, lauryl glucoside, and decyl glucoside, and more typically caprylyl/capryl glucoside and decyl glucoside.

The preferred alkyl poly glucosides are available under the trade name, for example:
For coco-glucoside: Plantacare® 818 UP sold by Cognis,
For decyl glucoside: Plantacare® 2000 UP sold by Cognis,
For caprylyl/capryl glucoside: Oramix™ CG 110 sold by Seppic,
For lauryl glucoside: Plantaren® 1200 N UP sold by Cognis,
For octyl glucoside: Rewosan sold by Rewo,
For sodium lauryl glucose carboxylate: Plantapon® LGC SORB.

Advantageously, the non-ionic surfactant is present in an amount ranging from about 0 wt. % to about 15 wt. %, preferably from about 1 wt. % to about 10 wt. %, or from about 2 wt. % to about 5 wt. %, relative to the total weight of the composition.

### Oil

As used herein, the term “oil” means a fatty compound or substance which is in the form of a liquid or a paste (non-solid) at room temperature (25° C.) under atmospheric pressure (760 mmHg). As the oil, those generally used in cosmetics can be used alone or in combination thereof.

The oil may be selected from oils of plant or animal origin, synthetic oils and mixtures thereof.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils or fats, mention may be made of, for example, lard oil, beef fat, mutton fat, chicken oil, snake oil, oviductus ranae, fish oil, horse fat, lanolin oil, and mixtures thereof.

As examples of synthetic oils, mention may be made of ester oils, ether oils, artificial triglycerides, hydrocarbon oils, silicone oils, and mixtures thereof.

The ester oils are preferably liquid esters of saturated or unsaturated, linear or branched C₁-C₂₆, preferably C₁₀-C₂₀, aliphatic monoacids or polyacids and of saturated or unsaturated, linear or branched C₁-C₂₆, preferably C₂-C₁₀ aliphatic monoalcohols or polyalcohols, the total number of carbon atoms of the esters being greater than or equal to 10.

Preferably, for the esters of monoalcohols, at least one among the alcohol and the acid from which the esters of the present invention are derived is branched.

Among the monoesters of monoacids and of monoalcohols, mention may be made of alkyl palmitates such as ethyl palmitate, ethylhexyl palmitate or isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis (2-ethylhexyl) sebacate; diisopropyl adipate; di-n-propyl adipate; dioctyl adipate; bis (2-ethylhexyl) adipate; diisostearyl adipate; bis (2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. The term “sugar” herein means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be selected especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, C₁-C₂₆ alkyl, preferably, C₁-C₁₀ alkyl oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri (2-ethylhexanoate), pentaerythrithyl tetra (2-ethylhexanoate), 2-ethylhexyl succinate, diethyl sebacate, and mixtures thereof.

As examples of artificial triglycerides, mention may be made of, for example, caprylyl/capryl triglyceride, glyceryl trimyristate, glyceryl tripalmitate, glyceryl trilinolenate, glyceryl trilaurate, glyceryl tricaprate, glyceryl tricaprylate, glyceryl tri (caprate/caprylate) and glyceryl tri (caprate/caprylate/linolenate).

An ether oil is an oil of formula R₁OR₂ in which R₁ and R₂ independently denote a linear, branched or cyclic C₄-C₂₄ alkyl group, preferably a C₆-C₁₈ alkyl group, and preferably a C₈-C₁₂ alkyl group. It may be preferable for R₁ and R₂ to be identical. Linear alkyl groups that may be mentioned include a butyl group, a pentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group.

Branched alkyl groups that may be mentioned include a 1-methylpropyl group, a 2-methylpropyl group, a t-butyl group, a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1, 1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group.

Cyclic alkyl groups that may be mentioned include a cyclohexyl group, a 3-methylcyclohexyl group and a 3,3,5-trimethylcyclohexyl group.

Advantageously, the ether oil is chosen from dicaprylyl ether, dicapryl ether, dilauryl ether, diisostearyl ether, dioctyl ether, nonyl phenyl ether, dodecyl dimethylbutyl ether, cetyl dimethylbutyl ether, cetyl isobutyl ether, and mixtures thereof.

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is selected from liquid polydialkylsiloxanes, especially liquid polydimethylsiloxanes (PDMS) (dimethicones) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly selected from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

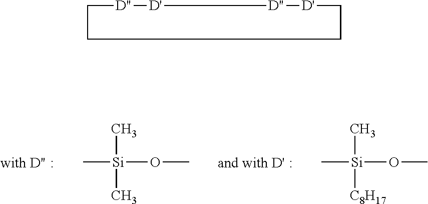

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis (2,2,2′,2′,3,3′-hexatrimethylsilyloxy) neopentane;
(ii) linear volatile polydialkylsiloxanes containing 2 to 9 silicon atoms and having a viscosity of less than or equal to 5×10-6 m2/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly selected from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the SilbioneR oils of the 47 and 70 047 series or the Mirasil oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm2/s;
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups are polydiarylsiloxanes, especially polydiphenylsiloxanes and polyalkylarylsiloxanes. Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be selected from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes, poly (C₆-20 olefin) and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosane, and decene/butene copolymer; and mixtures thereof.

In one embodiment, the composition according to the present invention comprises oils of plant or animal origin and/or ester oils, and optionally hydrocarbon oils and/or silicone oils.

In a preferred embodiment, the composition according to the present invention comprises oils of plant origin.

In a preferred embodiment, the composition according to the present invention comprises oils of plant origin, and C₂-C₁₀ alkyl palmitate and/or C₂-C₁₀ alkyl myristate, and especially sunflower oil, and ethylhexyl palmitate and/or isopropyl myristate.

In a preferred embodiment, the composition according to the present invention comprises oils of plant origin, and hydrocarbon oils and/or silicone oils; and especially sunflower oil, and dimethicones and/or isododecane.

In a preferred embodiment, the composition according to the present invention comprises ester oils, and hydrocarbon oils and/or silicone oils; and especially ethylhexyl palmitate and/or isopropyl myristate, and dimethicones and/or isododecane.

Advantageously, the oil is present in an amount of higher than about 10 wt. %, preferably ranging from about 25 wt. % to about 85 wt. %, or ranging from about 40 wt. % to about 70 wt. %, relative to the total weight of the composition.

Further, when the hydrocarbon oils and/or silicone oils are present, the amount of each of the hydrocarbon oil and silicone oil is equal to or lower than about 10 wt. %, preferably from about 0.5 wt. % to about 8 wt. %, or from about 1 wt. % to about 5 wt. %, relative to the total weight of the composition.

### Interface Stabilizer

The composition according to the present invention comprises at least one interface stabilizer.

As used herein, the term “interface stabilizer” means a substance capable of maintaining the stability of the interface between the water phase and the oil phase in an emulsified system, and preferably, the interface stabilizer can boost and stabilize the foams formed in the emulsified system.

In this aspect, the interface stabilizer according to the present invention plays a different role from a blowing agent, as a blowing agent is used to generate foams, e.g. a suitable surfactant, while said interface stabilizer is able to boost and stabilize the foams after their generation.

Preferably, the interface stabilizer according to the present invention is a polyurethane polymer. The polyurethane polymer is conventionally formed by the reaction of prepolymer (i) with a coreactant (ii), and optionally is terminated by an end-capping agent (iii).

The prepolymer (i) may be formed by the reaction of polyhydroxyl compounds, such as dihydroxyl compound or trihydroxyl compound, polyisocyanates, e.g. diisocyanates, and optionally low molecular weight diols which are optionally substituted with ionic groups or potential ionic groups.

Suitable polyhydroxyl compounds include those having at least two hydroxy groups, such as two or three hydroxy groups, and having number average molecular weights ranging from about 700 to about 16,000, such as, for example, from about 750 to about 5000. Non-limiting examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. In various embodiments, polyester polyols, polyether polyols, and polyhydroxy polycarbonates may be chosen. Mixtures of such compounds are also within the scope of the disclosure.

The polyester diol(s) may optionally be prepared from aliphatic, cycloaliphatic, or aromatic dicarboxylic or polycarboxylic acids, or anhydrides thereof; and dihydric or trihydric alcohols such as diols or triols chosen from aliphatic, alicyclic, or aromatic diols or triols.

The aliphatic dicarboxylic or polycarboxylic acids may be chosen from, for example, succinic, fumaric, glutaric, 2,2-dimethylglutaric, adipic, itaconic, pimelic, suberic, azelaic, sebacic, maleic, malonic, 2,2-dimethylmalonic, nonanedicarboxylic, decanedicarboxylic, dodecanedioic, 1,3-cyclohexanedicarboxylic, 1,4-cyclohexanedicarboxylic, 2,5-norboranedicarboxylic, diglycolic, thiodipropionic, 2,5-naphthalenedicarboxylic, 2,6-naphthalenedicarboxylic, phthalic, terephthalic, isophthalic, oxanic, o-phthalic, tetrahydrophthalic, hexahydrophthalic or trimellitic acid.

In some embodiments, the acid anhydrides may be chosen from o-phthalic, trimellitic or succinic acid anhydride or mixtures thereof. By way of non-limiting example only, the dicarboxylic acid may be adipic acid.

The dihydric alcohols may be chosen from ethanediol, ethylene glycol, diethylene glycol, triethylene glycol, trimethylene glycol, tetraethylene glycol, 1,2-propanediol, dipropylene glycol, tripropylene glycol, tetrapropylene glycol, 1,3-propanediol, 1,4-butanediol, 1,3-butanediol, 2,3-butanediol, 1,5-pentanediol, 1,6-hexanediol, 2,2-dimethyl-1,3-propanediol, 1,4-dihydroxycyclohexane, 1,4-dimethylolcyclohexane, cyclohexanedimethanol, 1,8-octanediol, 1,10-decanediol, 1,12-dodecanediol, neopentyl glycol, or mixtures thereof. The cycloaliphatic and/or aromatic dihydroxyl compounds may also be suitable as the dihydric alcohol(s) for the preparation of the polyester polyol(s). The trihydric alcohols may be chosen from alkylidine triols, such as iso-butanetriol, neo-pentanetriol, and neo-hexanetriol, e.g. trimethylolpropane.

The polyester polyols may also be chosen from homopolymers or copolymers of lactones, which are, in at least certain embodiments, obtained by addition reactions of lactones or lactone mixtures, such as γ-butyrolactone, γ-caprolactone, δ-caprolactone, ε-caprolactone and/or methyl-ε-caprolactone with the appropriate polyfunctional, e.g. difunctional or trifunctional, starter molecules such as, for example, the dihydric or trihydric alcohols mentioned above. The corresponding polymers of caprolactone may be chosen in at least some embodiments.

The polyester polyol may be obtained by polycondensation of dicarboxylic acids, such as adipic acid, with polyols, e.g. diols, such as hexanediol, neopentyl glycol, or mixtures thereof.

The polycarbonates containing hydroxyl groups comprise those known per se, such as the products obtained by reacting diols, such as (1,3)-propanediol, (1,4)-butanediol, and/or (1,6)-hexanediol, diethylene glycol, triethylene glycol, or tetraethylene glycol with diaryl carbonates, for example diphenyl carbonate or phosgene.

The polyether polyols may be obtained in any known manner by reacting starting compounds which contain reactive hydrogen atoms with alkylene oxides, such as, for example, ethylene oxide; propylene oxide; butylene oxide; styrene oxide; tetrahydrofuran; or epichlorohydrin, or with mixtures of these alkylene oxides. In certain embodiments, the polyethers do not contain more than about 10% by weight of ethylene oxide units. For example, polyethers obtained without addition of ethylene oxide may be chosen. Further, the above polyether polyols may contain a moiety derived from dimerized fatty alcohols, e.g. hydrogenated dilinoleyl alcohol.

Polyethers modified with vinyl polymers are also suitable according to various embodiments of the disclosure. Products of this type can be obtained by polymerization, for example, of styrene and acrylonitrile in the presence of polyethers.

Among the polythioethers which may be chosen include the condensation products obtained from thiodiglycol per se and/or with other glycols, dicarboxylic acids, formaldehyde, aminocarboxylic acids, and/or amino alcohols. The products obtained are either mixed polythioethers, polythioether esters, or polythioether ester amides, depending on the co-components.

The polyacetals include but are not limited to the compounds which can be prepared from aldehydes, for example formaldehyde, and from glycols, such as diethylene glycol, triethylene glycol, ethoxylated 4,4′-(dihydroxy) diphenyl-dimethylmethane, and (1,6)-hexanediol. Polyacetals useful according to various non-limiting embodiments of the disclosure can also be prepared by polymerization of cyclic acetals.

The polyhydroxy polyesteramides and polyamines include, for example, the mainly linear condensation products obtained from saturated or unsaturated, polybasic carboxylic acids or anhydrides thereof, and from saturated or unsaturated, polyvalent amino alcohols, from diamines, or from polyamines, as well as mixtures thereof.

The monomers for the production of polyacrylates having hydroxyl functionality comprise acrylic acid, methacrylic acid, crotonic acid, maleic anhydride, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, glycidyl acrylate, glycidyl methacrylate, 2-isocyanatoethyl acrylate, and 2-isocyanatoethyl methacrylate.

The polyisocyanates include, for example, organic diisocyanates having a molecular weight ranging from about 100 to about 1500, such as about 112 to about 1000, or about 140 to about 400.

The diisocyanates are those chosen from the general formula R₂ (NCO)₂, in which R₂ represents a divalent aliphatic hydrocarbon group comprising from about 4 to 18 carbon atoms, a divalent cycloaliphatic hydrocarbon group comprising from about 5 to 15 carbon atoms, or a divalent aromatic hydrocarbon group comprising from about 6 to 15 carbon atoms. Examples of the organic diisocyanates which may be chosen include, but are not limited to, tetramethylene diisocyanate, 1,6-hexamethylene diisocyanate, dodecamethylene diisocyanate, cyclohexane-1,3-diisocyanate and cyclohexane-1,4-diisocyanate, 1-isocyanato-3-isocyanatomethyl-3,5,5-trimethylcyclohexane (isophorone diisocyanate or IPDI), bis (4-isocyanatocyclohexyl)-methane, 1,3-bis (isocyanatomethyl)-cyclohexane and 1,4-bis (isocyanatomethyl) cyclohexane and bis (4-isocyanato-3-methylcyclohexyl) methane. Mixtures of diisocyanates can also be used.

In some embodiments, diisocyanates are chosen from aliphatic and cycloaliphatic diisocyanates. For example, 1,6-hexamethylene diisocyanate, isophorone diisocyanate, and dicyclohexylmethane diisocyanate, as well as mixtures thereof may be chosen.

The low molecular weight diols, may in at least certain embodiments allow a stiffening of the polymer chain. The expression “low molecular weight diols” means diols having a molecular weight ranging from about 50 to about 800, such as about 60 to 700, or about 62 to 200. They may, in various embodiments, contain aliphatic, alicyclic, or aromatic groups. In some embodiments, the compounds contain only aliphatic groups.

The diols that may be chosen may optionally have up to about 20 carbon atoms, and may be chosen, for example, from ethylene glycol, diethylene glycol, propane-1,2-diol, propane-1,3-diol, butane-1,4-diol, 1,3-butylene glycol, neopentyl glycol, butylethylpropanediol, cyclohexanediol, 1,4-cyclohexanedimethanol, hexane-1,6-diol, bisphenol A (2,2-bis (4-hydroxyphenyl) propane), hydrogenated bisphenol A (2,2-bis (4-hydroxycyclohexyl)-propane), or mixtures thereof.

Optionally, the low molecular weight diols may contain ionic or potentially ionic groups. Suitable low molecular weight diols containing ionic or potentially ionic groups may be chosen from those disclosed in U.S. Pat. No. 3,412,054. In various embodiments, compounds may be chosen from dimethylolbutanoic acid (DMBA), dimethylolpropionic acid (DMPA), and carboxyl-containing caprolactone polyester diol. If low molecular weight diols containing ionic or potentially ionic groups are chosen, they may, for example, be used in an amount such that less than about 0.30 meq of —COOH is present per gram of polyurethane in the polyurethane dispersion. In some embodiments, the low molecular weight diols containing ionic or potentially ionic groups are not used.

The coreactants (ii) are compounds containing functional groups such as hydroxyl or amine groups, adapted to react with isocyanate groups in preference to the carboxyl group.

Specifically, the coreactants (ii) are aliphatic or cycloaliphatic or aromatic hydrocarbons substituted with at least two hydroxyl or amine groups, and optionally substituted with ionic groups or potentially ionic groups. In various embodiments, compounds may optionally be chosen from alkylene diamines, such as hydrazine, ethylenediamine, propylenediamine, 1,4-butylenediamine, and piperazine. In various embodiments, compounds may be chosen from alkylene diols, such as ethylene glycol, 1,4-butanediol (1,4-BDO or BDO), 1,6-hexanediol.

As used herein, ionic or potentially ionic groups may include groups comprising ternary or quaternary ammonium groups, groups convertible into such groups, carboxyl groups, carboxylate groups, sulphonic acid groups, and sulphonate groups. At least partial conversion of the groups convertible into salt groups of the type mentioned may take place before or during the mixing with water. Special compounds may be chosen from dimethylolbutanoic acid (DMBA), dimethylolpropionic acid (DMPA), or carboxyl functional polyester comprising excess equivalents of dicarboxylic acid reacted with lesser equivalents of glycol or carboxyl-containing caprolactone polyester diol.

The end-capping agent (iii) can be derived from compounds having the formula: R—NH—R′, wherein R represents a hydrogen atom or an alkylene radical optionally having a hydroxyl end and R′ represents an alkylene radical optionally having a hydroxyl end.

Suitable end-capping agent includes compounds such as monoamines, especially secondary monoamines or monoalcohols. Examples include: methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl (methyl) aminopropylamine, morpholine, piperidine, diethanolamine and suitable substituted derivatives thereof, amide-amines of primary diamines and monocarboxylic acids, monocetimes of primary diamines, primary/tertiary amines such as N,N-dimethylamino-propylamine and the like. The end-capping agent can be chosen from C₁-C₁₀ alcohols, such as methanol, butanol, hexanol, 2-ethylhexyl alcohol, isodecyl alcohol, and mixtures thereof, or chosen from fatty alcohols, such as stearyl alcohol, cetanol, eicosanol, and mixtures thereof, and amino alcohols such as aminomethylpropanol (AMP) are also suitable.

By way of non-limiting example, the polyurethane polymer includes, but are not limited to, isophthalic acid/adipic acid/hexylene glycol/neopentyl glycol/dimethylolpropanoic acid/isophorone diisocyanate copolymer (INCI name: Polyurethane-1, such as LUVISET® P.U.R, BASF), a copolymer of hexylene glycol, neopentyl glycol, adipic acid, saturated methylene diphenyldiisocyanate and dimethylolpropanoic acid monomers (INCI name: Polyurethane-2), a copolymer of PPG-17, PPG-34, isophorone diisocyanate and dimethylolpropanoic acid monomers (INCI name: Polyurethane-4), a copolymer of isophthalic acid, adipic acid, hexylene glycol, neopentyl glycol, dimethylolpropanoic acid, isophorone diisocyanate and bis-ethylaminoisobutyl-dimethicone monomers (INCI name: Polyurethane-6), Isophorone diisocyanate, cyclohexanedimethanol, dimethylol butanoic acid, polyalkylene glycol and N-methyl diethanolamine copolymer (INCI name: Polyurethane-10), trimethylolpropane, neopentyl glycol, dimethylol propionic acid, polytetramethylene ether glycol and isocyanato methylethylbenzene copolymer (INCI name: Polyurethane-12), isophorone diisocyanate, dimethylol propionic acid, and 4,4′-isopropylidenediphenol reacted with propylene oxide, ethylene oxide and PEG/PPG-17/3 copolymer (INCI name: Polyurethane-14), isophorone diisocyanate, adipic acid, triethylene glycol and dimethylolpropionic acid copolymer (INCI name: Polyurethane-15), 2-methyl-2,4-pentanediol, polymer with 2,2-dimethyl-1,3-propanediol, hexanedioic acid, methylenedicyclohexanediisocyanate and 2,2-di (hydroxymethyl) propanoic acid, hydrolysed, tris (2-hydroxyethyl) amine salts, reaction products with 1,2-ethanediamine (INCI name: Polyurethane-17), a complex polymer that is formed by the reaction of polyperfluoroethoxymethoxy difluorohydroxyethyl ether and isophorone diisocyanate (IPDI) to form a prepolymer, the prepolymer being further reacted with the triethylamine salt of 3-hydroxy-2-(hydroxymethyl)-2-methyl-1-propionic acid (INCI name: Polyurethane-27), a complex polymer formed by reacting dimethylolpropionic acid and a polyester composed of adipic acid, hexylene glycol, neopentyl glycol with methylene dicyclohexyldiisocyanate (SMDI) to form a prepolymer, which is neutralized with triethylamine and then chain-extended with hydrazine (INCI name: Polyurethane-33); those sold under the tradename Baycusan® by Bayer such as, Baycusan® C₁₀₀₀ (INCI name: Polyurethane-34), Baycusan® C₁₀₀₁ (INCI name: Polyurethane-34), Baycusan® C₁₀₀₃ (INCI name: Polyurethane-32), Baycusan® C₁₀₀₄ (INCI name: Polyurethane-35), Baycusan® C₁₀₀₈ (INCI name: Polyurethane-48), a copolymer formed by reacting hydrogenated polybutanediol, 1,6-hexamethylene diisocyanate, hydrogenated dilinoleyl alcohol, and 1,4-butanediol, and is capped with stearyl Alcohol, under the trade name of Oilkemia™ 5S Polymer available from Lubrizol Corporation (INCI name: Polyurethane-79), and Oilkemia™ 5S CC Polymer available from Lubrizol Corporation (INCI name: HDI/TRIMETHYLOL HEXYLLACTONE CROSSPOLYMER).

In one embodiment, the polyhydroxyl compounds are polyester polyols, and preferably, homopolymers or copolymers of lactones, such as γ-butyrolactone, y-caprolactone, 8-caprolactone, ¿-caprolactone and/or methyl-¿-caprolactone, with dihydric or trihydric alcohols, such as butanediol and trimethylolpropane.

In another embodiment, the polyhydroxyl compounds are polyether polyols, preferably obtained by reacting starting compounds which contain reactive hydrogen atoms with alkylene oxides, e.g. butylene oxide, and especially the polyether polyols containing a moiety derived from dimerized fatty alcohols, e.g. hydrogenated dilinoleyl alcohol.

In one embodiment, the polyisocyanate is selected from the diisocyanate containing aliphatic, cycloaliphatic and aromatic hydrocarbon group, especially, 1,6-hexamethylene diisocyanate, isophorone diisocyanate, and dicyclohexylmethane diisocyanate.

In one embodiment, the coreactant is chosen from compounds containing functional groups such as hydroxyl or amine groups, adapted to react with isocyanate groups in preference to the carboxyl group, and preferably is chosen from alkylene diamines, such as hydrazine, ethylenediamine, propylenediamine, 1,4-butylenediamine, piperazine, and alkylene diols, such as ethylene glycol, 1,4-butanediol, and 1,6-hexanediol.

In one embodiment, the end-capping agent is selected from monoamines, alkanolamines, amide-amines, C₁-C₁₀ alcohols, and fatty alcohols, such as stearyl alcohol, and mixtures thereof

According to the present invention, the especially preferable polyurethane polymers are HDI/trimethylol hexyllactone crosspolymer; and a copolymer formed by reacting hydrogenated polybutanediol, 1,6-hexamethylene diisocyanate, hydrogenated dilinoleyl alcohol, and 1,4-butanediol, and is capped with stearyl alcohol; and for example, the products under the trade names Oilkemia™ 5S CC POLYMER and Oilkemia™ 5S POLYMER available from Lubrizol Corporation.

Advantageously, the interface stabilizer is present in an amount ranging from about 0.01 wt. % to about 10 wt. %, preferably from about 0.05 wt. % to about 5 wt. %, or from about 0.1 wt. % to about 2 wt. %, relative to the total weight of the composition.

Further, the interface stabilizer according to the present invention may provide improved rheology, thickening efficiency, clarity, and non-tacky feel for the product comprising the same. In other words, the interface stabilizer according to the present invention also serves as a thickener or gelling agent. Conventionally, the thickeners or gelling agents can also selected from amides, e.g. Polyamide-8 or Cocamide MEA; acrylates, such as Poly C₁₀-30 Alkyl Acrylate or Acrylates/C₁₀-30 Alkyl Acrylate Crosspolymer; gums, e.g. Xanthan gum; cellulose derivatives, e.g. hydroxypropyl cellulose; and silicone crosspolymers, e.g. vinyl dimethicone crosspolymer.

However, the traditional thickeners or gelling agents, such as polyamides, acrylates and gums, cannot play a role in boosting and stabilizing the foams formed in the emulsified system, and some of them even worsen the foaming performances of the emulsified system. Surprisingly, the inventor discovers that the interface stabilizer according to the present invention not only provides improved rheology, thickening efficiency, clarity, and non-tacky feel, but also boosts and stabilizes the foams formed in the emulsified system. By means of the interface stabilizer according to the present invention, the foaming performances may improve more than about 20%, more than about 50%, and even up to about 100%, relative to the emulsified system which does not comprise such an interface stabilizer, wherein the term “foaming performance” has a common meaning as in the cosmetic field, including but not limited to the volume, density and stability of the foams in a emulsified system.

### Aqueous Phase

Generally, the composition according to the present invention comprises at least one aqueous phase.

The aqueous phase of the composition according to the present invention comprises water and optionally one or more water-miscible or at least partially water-miscible compounds, for instance C₂ to C₈ lower polyols or monoalcohols, such as ethanol and isopropanol.

The term “polyol” should be understood as any organic molecule comprising at least two free hydroxyl groups. Examples of polyols that may be mentioned include glycols, for instance butylene glycol, propylene glycol, and isoprene glycol, caprylyl glycol, glycerol (i.e. glycerin) and polyethylene glycols.

The aqueous phase may represent from about 20 wt. % to about 80 wt. %, preferably from about 20 wt. % to about 60 wt. %, or from about 30 wt. % to about 60 wt. %, relative to the total weight of the composition.

### Other Ingredients

The composition according to the present invention may also comprise other ingredients, known previously elsewhere in cosmetic compositions, such as cosmetic active ingredient, for example hydroxyacetophenone, and various common adjuvants, for example, sequestering agents such as EDTA and etidronic acid, preserving agents such as phenoxyethanol and salicylic acid, opacifiers, fragrances, and so on.

### Combination

The combination according to the present invention comprises, substantially consists of, or even consists of a surfactant system and at least one interface stabilizer, for improving foam properties, even for high-oil products. Specifically, the surfactant system comprises at least one alkyl sulfosuccinate as the anionic surfactant and at least one amphoteric surfactant, and optionally at least one nonionic surfactant, as mentioned above.

Preferably, the alkyl sulfosuccinate is selected from the mono- or di-alkyl sulfosuccinates, the amphoteric surfactant is selected from the (C₈-C₂₀) alkylamido (C₁-C₆) alkylbetaines, and the nonionic surfactant is selected from the alkyl poly glucoside, wherein the alkyl sulfosuccinate, the amphoteric surfactant and the nonionic surfactant and the amounts thereof are defined as above. Further, the interface stabilizer and the amounts thereof are defined as above.

In one embodiment, the present invention discloses a combination of improving foam properties of high-oil products, comprising a surfactant system consisting of at least one mono-alkyl sulfosuccinate, at least one di-alkyl sulfosuccinate and at least one (C₈-C₂₀) alkylamido (C₁-C₆) alkylbetaine, and an interface stabilizer which is a polyurethane polymer; preferably, the present invention discloses a combination of improving foam properties of high-oil products, comprising a surfactant system consisting of cocamidopropyl betaine, disodium lauryl sulfosuccinate and diethylhexyl sodium sulfosuccinate, and a polyurethane polymer as mentioned above.

In another embodiment, the present invention discloses a combination of improving foam properties of high-oil products, comprising a surfactant system consisting of at least one mono-alkyl sulfosuccinate and at least one (C₈-C₂₀) alkylamido (C₁-C₆) alkylbetaine, and an interface stabilizer which is a polyurethane polymer; preferably, the present invention discloses a combination of improving foam properties of high-oil products, comprising a surfactant system consisting of cocamidopropyl betaine and disodium lauryl sulfosuccinate, and a polyurethane polymer as mentioned above.

In still another embodiment, the present invention discloses a combination of improving foam properties of high-oil products, comprising a surfactant system consisting of at least one di-alkyl sulfosuccinate, at least one alkyl poly glucoside and at least one (C₈-C₂₀) alkylamido (C₁-C₆) alkylbetaine, and an interface stabilizer which is a polyurethane polymer; preferably, the present invention discloses a combination of improving foam properties of high-oil products, comprising a surfactant system consisting of cocamidopropyl betaine, caprylyl/capryl glucoside and diethylhexyl sodium sulfosuccinate, and a polyurethane polymer as mentioned above.

As used herein, the term “high-oil” means that the total amount of oils in a product represents higher than about 10 wt. %, and even higher than about 40 wt. %, relative to the total weight of the product.

The “product” in the term “high-oil product” is preferably in the form of emulsion, lotion, balm or cream, is preferably used for cleansing and/or removing makeup from keratin materials, especially the facial skin.

So far, for cleansing products, the foam properties are heavily impact by the oil content therein, and a high-oil product normally has a very poor foaming property. By means of the combination according to the present invention, good to excellent foaming properties can be obtained even at a very high oil content, e.g. an oil content of higher than about 10 wt. %, and even higher than about 40 wt. %, relative to the total weight of the product.

### Foaming Composition

The foaming composition according to the present invention comprises a surfactant system, at least one oil, an interface stabilizer, and an optional aqueous phase, especially for cleansing and/or removing makeup from keratin materials, especially facial skin, and preferably in the form of emulsion, lotion, balm or cream.

Specifically, the surfactant system comprises at least one alkyl sulfosuccinate as the anionic surfactant and at least one amphoteric surfactant, and optionally at least one nonionic surfactant. The surfactant system, each surfactant therein and the amounts of the surfactant system and each surfactant are defined as above.

The at least one oil is selected from oils of plant or animal origin, ester oils, ether oils, hydrocarbon oils, silicone oils and mixtures thereof; and the interface stabilizer is chosen from a polyurethane polymer. The at least one oil, the interface stabilizer and the amounts thereof are defined as above.

The aqueous phase comprises water and optionally at least one monoalcohol and/or polyol as defined above.

Alternatively, the foaming composition according to the present invention comprises the above combination and at least one oil, wherein both the combination and the at least one oil are defined as above.

The weight ratio of the polyurethane polymer to the at least one oil ranges from about 1:500 to about 1:1, preferably from about 1:200 to about 1:10, or from about 1:100 to about 1:25.

The composition comprising an amount of lower than the above ranges of the polyurethane polymer cannot obtain desirable foaming performances, and the composition comprising an amount of higher than the above ranges of the polyurethane polymer may rather inhibit the boosting of foams.

By means of the specific components and the specific weight ratio of the polyurethane polymer to the at least one oil, the foaming composition according to the present invention provides effective cleansing of the keratin materials, perceived and desirable foams, and no irritation to eyes and the keratin materials, rendering the keratin materials fresh and clean.

### Method and Use

The composition according to the present invention can be used for a process for cleansing and/or removing makeup from keratin materials, such as the skin, in particular the face, by being applied to the keratin materials.

The composition according to the invention may be applied by any means enabling a uniform distribution, in particular using a finger, or a cotton ball, a rod, a brush, gauze, or a spatula, and can be removed by rinsing with water.

Thus, according to another aspect, the present invention relates to a method for cleansing and/or removing makeup from keratin materials, in particular the skin, comprising application to the keratin materials, in particular the skin, of the composition according to the present invention, and rinsing off said composition after an optional period of time.

According to still another aspect, the present invention relates to use of the combination according to the present invention for improving foam properties, even for high-oil products.

According to further another aspect, the present invention relates to use of a polyurethane polymer, especially that is formed by the reaction of prepolymer (i) with a coreactant (ii), and optionally is terminated by an end-capping agent (iii), wherein the prepolymer (i) is formed by the reaction of polyhydroxyl compounds, polyisocyanates, and optionally low molecular weight diols, for improving rheology of an emulsified system and boosting and stabilizing the foams formed therein.

The present invention is illustrated in greater detail by the examples described below, which are given as non-limiting illustrations.

### EXAMPLES

Main raw materials used, trade names and supplier thereof were listed in Table 1.

TABLE 1

| INCI NAME | TRADE NAME | SUPPLIER |
|---|---|---|
| DIETHYLHEXYL SODIUM | TEGO | EVONIK |
| SULFOSUCCINATE | SULFOSUCCINATE | GOLDSCHMI |
| | DO 75 | |
| DISODIUM LAURYL SULFOSUCCINATE | FS401P | GUANGZHOU |
| | | FLOWER'S SONG |
| | | FINE CHEMIC |
| COCAMIDOPROPYL BETAINE | DEHYTON PK 45 | BASF |
| CAPRYLYL/CAPRYL GLUCOSIDE | ORAMIX CG 110L | SEPPIC |
| CAPRYLIC/CAPRIC TRIGLYCERIDE (and) | OILKEMIA 5S CC | LUBRIZOL |
| HYDROGENATED POLY(C6-20 OLEFIN) | POLYMER | |
| (and) HDI/TRIMETHYLOL | | |
| HEXYLLACTONE CROSSPOLYMER | | |
| POLYURETHANE-79 (and) | OILKEMIA 5S | LUBRIZOL |
| CAPRYLIC/CAPRIC TRIGLYCERIDE | POLYMER | |
| HELIANTHUS ANNUUS (SUNFLOWER) | REFINED | AAK KAMANI |
| SEED OIL | SUNFLOWER OIL | PRIVATE |
| ETHYLHEXYL PALMITATE | CEGESOFT C 24 | BASF |
| ISOPROPYL MYRISTATE | ISOPROPYL- | BASF |
| | MYRISTATE | |
| DIMETHICONE | KF-96 A-6CS | SHIN ETSU |
| ISODODECANE | ISODODECANE | INEOS |
| XANTHAN GUM | KELTROL CG-T | CP KELCO |
| POLY C10-30 ALKYL ACRYLATE | TEGO SP 13-6 | EVONIK MATERIALS |
| | | NETHERLANDS B.V. |
| ACRYLATES/C10-30 ALKYL ACRYLATE | PEMULEN TR-2 | LUBRIZOL |
| CROSSPOLYMER | POLYMER | |
| POLYAMIDE-8 | OLEOCRAFT LP-20- | CRODA |
| | PA-(MV) | |

### Inventive Examples 1, 1′ and 2 and Comparative Examples 1-5

The compositions according to inventive formulas IEs. 1, 1′ and 2 and comparative formulas CEs. 1-5 were prepared with the ingredients listed in Table 2 (the contents were expressed as weight percentages of ingredients with regard to the total weight of each 5 composition, unless otherwise indicated):

TABLE 2

| COMPONENTS | IE. 1′ | IE. 1 | IE. 2 | CE. 1 | CE. 2 | CE. 3 | CE. 4 | CE. 5 |
|---|---|---|---|---|---|---|---|---|
| WATER | QS | QS | QS | QS | QS | QS | QS | QS |
| COCAMIDOPROPYL BETAINE | 2.147 | 2.147 | 2.147 | 2.147 | 2.147 | 2.147 | 2.147 | 2.147 |
| GLYCERIN | 2 | 2 | 2 | 2 | 2 | 2 | 2 | 2 |
| CAPRYLYL/CAPRYL GLUCOSIDE | 2.208 | 2.208 | 2.208 | 2.208 | 2.208 | 2.208 | 2.208 | 2.208 |
| CAPRYLIC/CAPRIC | | 0.5 | 1 | | | | | |
| TRIGLYCERIDE (and) | | | | | | | | |
| HYDROGENATED POLY(C6-20 | | | | | | | | |
| OLEFIN) (and) HDI/TRIMETHYLOL | | | | | | | | |
| HEXYLLACTONE | | | | | | | | |
| CROSSPOLYMER | | | | | | | | |
| POLYURETHANE-79 (and) | 0.5 | | | | | | | |
| CAPRYLIC/CAPRIC | | | | | | | | |
| TRIGLYCERIDE | | | | | | | | |
| POLYAMIDE-8 | | | | | 0.5 | | | |
| ACRYLATES/C10-30 ALKYL | | | | | | 0.5 | | |
| ACRYLATE CROSSPOLYMER | | | | | | | | |
| POLY C10-30 ALKYL ACRYLATE | | | | | | | 0.5 | |
| XANTHAN GUM | | | | | | | | 0.5 |
| HELIANTHUS ANNUUS | 50 | 50 | 50 | 50 | 50 | 50 | 50 | 50 |
| (SUNFLOWER) SEED OIL | | | | | | | | |
| DIETHYLHEXYL SODIUM | 3.488 | 3.488 | 3.488 | 3.488 | 3.488 | 3.488 | 3.488 | 3.488 |
| SULFOSUCCINATE | | | | | | | | |

### Preparation Procedure:

The compositions were prepared as follows:
1) mixing water, cocamidopropyl betaine, glycerin and caprylyl/capryl glucoside at 65° C. in main kettle to obtain Pre-mix 1,
2) mixing oil/oil mixture with Caprylic/Capric Triglyceride (and) Hydrogenated Poly (C₆₋₂₀olefin) (and) HDI/Trimethylol Hexyllactone Crosspolymer or Polyurethane-79 or Polyamide-8 or PolyC₁₀₋₃₀ Alkyl Acrylate at 90° C. in annex under stirring to obtain Pre-mix 2, and
3) mixing Pre-mix 1 with Pre-mix 2, and cooling down to RT, then adding Diethylhexyl Sodium Sulfosuccinate; or
1′) mixing water, cocamidopropyl betaine, glycerin, caprylyl/capryl glucoside and Acrylates/C₁₀-30 Alkyl Acrylate Crosspolymer or Xanthan gum at 65° C. in main kettle to obtain Pre-mix 1,
2′) adding oil/oil mixture into annex at RT to obtain Pre-mix 2, and
3′) mixing Pre-mix 1 with Pre-mix 2, and cooling down to RT, then adding Diethylhexyl Sodium Sulfosuccinate.

### Evaluation of Foaming Performances

Preparation procedure:
1) taking 0.5 ml of each composition to be tested with a syringe, and 3×1 ml of water with pipettes;
2) rinsing hands under tap water for 2 seconds and placing the 0.5 ml of product to palm with the syringe, then adding 1 ml of water with one pipette;
3) making back and forth movements for 20 rounds (2 circles/second) and pausing to gather foam in the palm to prevent foam from flowing if necessary;
4) adding 1 ml of water with one pipette again, and making back and forth movements for 20 rounds (2 circles/second), and pausing to gather foam in the palm to prevent foam from flowing if necessary; and
5) putting together all the foam in one palm.

Foaming property was evaluated by the volume and density of foam obtained at the end of rubbing hands (40 rounds), regardless of size of bubbles. A score within a range of 0-15 was given, wherein 0 meant the property of foam was zero, 1-4 meant low foam property, 5-7 meant middle foam property, 8-11 meant good foam property, and 12-15 meant excellent foam property.

Foaming stability was evaluated by the volume of foam obtained after 30s. A score within a range of 0-15 was given, wherein 0 meant the property of foam was zero, 1-4 meant low foam property, 5-7 meant middle foam property, 8-11 meant good foam property, and 12-15 meant excellent high foam property.

The results were summarized in Table 3.

TABLE 3

| Properties | IE. 1′ | IE. 1 | IE. 2 | CE. 1 | CE. 2 | CE. 3 | CE. 4 | CE. 5 |
|---|---|---|---|---|---|---|---|---|
| Foaming | 14 | 14 | 12 | 9 | 4 | 2 | 1 | 2 |
| properties | | | | | | | | |
| Foaming | 12 | 12 | 12 | 6 | 3 | 1 | 1 | 1 |
| stability | | | | | | | | |

From the above Tables 2-3, it could be seen that the compositions, i.e. IE. 1, IE. 1′ and IE. 2, comprising the specific interface stabilizer according to the present invention indeed obtained excellent foaming performances, including both the foaming properties and the foaming stability, since all of them obtained scores of 12 or higher.

By contrast, the compositions comprising the traditional thickeners, e.g., those comprised in CEs. 2-5, did not provide improved foaming performances, and even deteriorating the foaming performances relative to the composition of CE. 1, which did not comprise the thickeners comprised in CEs. 2-5 or the interface stabilizer according to the present invention. However, the interface stabilizer of the present invention not only provided improved rheology and thickening efficiency of compositions, as known by those skilled in the art, but also boosted and stabilized the foams formed therein.

### Inventive Examples 1, 3, 4 and 4′

The compositions according to inventive formulas IEs. 1, 3, 4 and 4′ were prepared with the ingredients listed in Table 4 (the contents were expressed as weight percentages of ingredients with regard to the total weight of each composition, unless otherwise indicated):

TABLE 4

| COMPONENTS | IE. 1 | IE. 3 | IE. 4 | IE. 4′ |
|---|---|---|---|---|
| WATER | QS | QS | QS | QS |
| COCAMIDOPROPYL BETAINE | 2.147 | 2.147 | 2.147 | 2.147 |
| GLYCERIN | 2 | 2 | 2 | 2 |
| CAPRYLIC/CAPRIC | 0.5 | 0.5 | 0.5 | |
| TRIGLYCERIDE (and) | | | | |
| HYDROGENATED POLY(C6- | | | | |
| 20 OLEFIN) (and) | | | | |
| HDI/TRIMETHYLOL | | | | |
| HEXYLLACTONE | | | | |
| CROSSPOLYMER | | | | |
| POLYURETHANE-79 (and) | | | | 0.5 |
| CAPRYLIC/CAPRIC | | | | |
| TRIGLYCERIDE | | | | |
| CAPRYLYL/CAPRYL | 2.208 | | | |
| GLUCOSIDE | | | | |
| DISODIUM LAURYL | | 3.5 | 3.5 | 3.5 |
| SULFOSUCCINATE | | | | |
| DIETHYLHEXYL SODIUM | 3.488 | 3.488 | 0 | 0 |
| SULFOSUCCINATE | | | | |
| HELIANTHUS ANNUUS | 50 | 50 | 50 | 50 |
| (SUNFLOWER) SEED OIL | | | | |

Preparation procedure:

The compositions were prepared as follows:
1) mixing water, cocamidopropyl betaine, glycerin and caprylyl/capryl glucoside, if present, at 65° C. in main kettle to obtain Pre-mix 1;
2) mixing oil/oil mixture with Caprylic/Capric Triglyceride (and) Hydrogenated Poly (C₆-20olefin) (and) HDI/Trimethylol Hexyllactone Crosspolymer or Polyurethane-79 at 90° C. in annex under stirring to obtain Pre-mix 2; and
3) mixing Pre-mix 1 with Pre-mix 2, and cooling down to RT, then adding Disodium Lauryl Sulfosuccinate and/or Diethylhexyl Sodium Sulfosuccinate.

### Evaluation of Foaming Performances

The foaming property and foaming stability of the compositions obtained were evaluated as described above.

The results were summarized in Table 5.

TABLE 5

| Properties | IE. 1 | IE. 3 | IE. 4 | IE. 4′ |
|---|---|---|---|---|
| Foaming properties | 14 | 10 | 12 | 12 |
| Foaming stability | 12 | 10 | 11 | 11 |

From the above Tables 4-5, it could be seen that the compositions, i.e. IEs. 1, 3, 4, and 4′ comprising the specific combination of the surfactant system and interface stabilizer according to the present invention indeed obtained good to excellent foaming performances, including both the foaming properties and the foaming stability, even at a high content of 50 wt. % of the oil, since all of them obtained scores of higher than 8.

### Inventive Examples 1, 5, 5′, 6, 6′, 7 and 7′

The compositions according to inventive formulas IEs. 1, 5, 5′, 6, 6′, 7 and 7′ were prepared with the ingredients listed in Table 6 (the contents were expressed as weight percentages of ingredients with regard to the total weight of each composition, unless otherwise indicated):

TABLE 6

| COMPONENTS | IE. 1 | IE. 5 | IE. 5′ | IE. 6 | IE. 6′ | IE. 7 | IE. 7′ |
|---|---|---|---|---|---|---|---|
| WATER | QS | QS | QS | QS | QS | QS | QS |
| COCAMIDOPROPYL | 2.147 | 2.147 | 2.147 | 2.147 | 2.147 | 2.147 | 2.147 |
| BETAINE | | | | | | | |
| GLYCERIN | 2 | 2 | 2 | 2 | 2 | 2 | 2 |
| CAPRYLYL/CAPRYL | 2.208 | 2.208 | 2.208 | 2.208 | 2.208 | 2.208 | 2.208 |
| GLUCOSIDE | | | | | | | |
| DIETHYLHEXYL SODIUM | 3.488 | 3.488 | 3.488 | 3.488 | 3.488 | 3.488 | 3.488 |
| SULFOSUCCINATE | | | | | | | |
| CAPRYLIC/CAPRIC | 0.5 | 2 | | 0.5 | | 1 | |
| TRIGLYCERIDE (and) | | | | | | | |
| HYDROGENATED | | | | | | | |
| POLY(C6-20 OLEFIN) (and) | | | | | | | |
| HDI/TRIMETHYLOL | | | | | | | |
| HEXYLLACTONE | | | | | | | |
| CROSSPOLYMER | | | | | | | |
| POLYURETHANE-79 (and) | | | 2 | | 0.5 | | 1 |
| CAPRYLIC/CAPRIC | | | | | | | |
| TRIGLYCERIDE | | | | | | | |
| HELIANTHUS ANNUUS | 50 | 30 | 30 | 39 | 39 | | |
| (SUNFLOWER) SEED OIL | | | | | | | |
| ISOPROPYL MYRISTATE | | 20 | 20 | | | | |
| ETHYLHEXYL | | | | | | 35 | 35 |
| PALMITATE | | | | | | | |
| ISODODECANE | | | | | | 5 | 5 |
| DIMETHICONE | | | | 1 | 1 | | |

### Preparation Procedure:

The compositions were prepared as follows:
1) mixing water, cocamidopropyl betaine, glycerin and caprylyl/capryl glucoside at 65° C. in main kettle to obtain Pre-mix 1;
2) mixing oil/oil mixture with Caprylic/Capric Triglyceride (and) Hydrogenated Poly (C₆-20olefin) (and) HDI/Trimethylol Hexyllactone Crosspolymer or Polyurethane-79 at 90° C. in annex under stirring to obtain Pre-mix 2; and
3) mixing Pre-mix 1 with Pre-mix 2, and cooling down to RT, then adding Diethylhexyl Sodium Sulfosuccinate.

### Evaluation of Foaming Performances

The foaming property and foaming stability of the compositions obtained were evaluated as described above.

The results were summarized in Table 7.

TABLE 7

| Properties | IE. 1 | IE. 5 | IE. 5′ | IE. 6 | IE. 6′ | IE. 7 | IE. 7′ |
|---|---|---|---|---|---|---|---|
| Foaming properties | 14 | 12 | 12 | 9 | 9 | 8 | 8 |
| Foaming stability | 12 | 11 | 11 | 8 | 8 | 8 | 8 |

From the above Tables 6-7, it could be seen that the compositions, i.e. IEs. 1, 5, 5′, 6, 6′, 7 and 7′, comprising the specific oil/oil mixture according to the present invention indeed obtained good to excellent foaming performances, including both the foaming properties and the foaming stability, since all of them obtained scores of 8 or higher.

### Inventive Examples 1, 8 and 8′ and Comparative Example 6

The compositions according to inventive formulas IEs. 1, 8 and 8′ and comparative formula CE. 6 were prepared with the ingredients listed in Table 8 (the contents were expressed as weight percentages of ingredients with regard to the total weight of each composition, unless otherwise indicated):

TABLE 8

| COMPONENTS | IE. 1 | IE. 8 | IE. 8′ | CE.6 |
|---|---|---|---|---|
| WATER | QS | QS | QS | QS |
| COCAMIDOPROPYL BETAINE | 2.147 | 2.147 | 2.147 | 2.147 |
| GLYCERIN | 2 | 2 | 2 | 2 |
| CAPRYLYL/CAPRYL GLUCOSIDE | 2.208 | 2.208 | 2.208 | 2.208 |
| DIETHYLHEXYL SODIUM | 3.488 | 3.488 | 3.488 | 3.488 |
| SULFOSUCCINATE | | | | |
| CAPRYLIC/CAPRIC | 0.5 | 0.2 | | |
| TRIGLYCERIDE (and) | | | | |
| HYDROGENATED POLY(C6-20 | | | | |
| OLEFIN) (and) HDI/TRIMETHYLOL | | | | |
| HEXYLLACTONE | | | | |
| CROSSPOLYMER | | | | |
| POLYURETHANE-79 (and) | | | 0.2 | |
| CAPRYLIC/CAPRIC | | | | |
| TRIGLYCERIDE | | | | |
| HELIANTHUS ANNUUS | 50 | 10 | 10 | 10 |
| (SUNFLOWER) SEED OIL | | | | |

### Preparation Procedure:

The compositions were prepared as follows:
1) mixing water, cocamidopropyl betaine, glycerin and caprylyl/capryl glucoside at 65° C. in main kettle to obtain Pre-mix 1;
2) mixing oil/oil mixture with Caprylic/Capric Triglyceride (and) Hydrogenated Poly (C₆-20olefin) (and) HDI/Trimethylol Hexyllactone Crosspolymer or Polyurethane-79, if present, at 90° C. in annex under stirring to obtain Pre-mix 2; and
3) mixing Pre-mix 1 with Pre-mix 2, and cooling down to RT, then adding Diethylhexyl Sodium Sulfosuccinate.

### Evaluation of Foaming Performances

The foaming property and foaming stability of the compositions obtained were evaluated as described above.

The results were summarized in Table 9.

TABLE 9

| Properties | IE.1 | IE.8 | IE.8′ | CE.6 |
|---|---|---|---|---|
| Foaming properties | 14 | 10 | 10 | 10 |
| Foaming stability | 12 | 9 | 9 | 6 |

From the above Tables 8-9, it could be seen that the compositions, i.e. IEs. 1, 8 and 8′, comprising a specific weight ratio of the interface stabilizer to the oil/oil mixture according to the present invention indeed obtained good to excellent foaming performances, including both the foaming properties and the foaming stability, since all of them obtained scores of higher than 8.

By contrast, the composition, not comprising the interface stabilizer of the present invention, i.e. CE. 6, merely obtained middle foam performance for the foaming stability, since it merely obtained a score of 6. It could be seen that the specific interface stabilizer of the present invention indeed brought improved foaming performances for the composition, as the composition of IE. 8 and 8′, having the same amount of the oil, obtained better foaming stability than the composition of CE. 6.

1. A foaming composition, comprising:
(a) at least one oil,
(b) at least one polymeric interface stabilizer of improving rheology of the composition and boosting and stabilizing the foams formed therein, and
(c) a surfactant system, comprising,
at least one alkyl sulfosuccinate,
at least one amphoteric surfactant, and
optionally, at least one nonionic surfactant.

2. The foaming composition according to claim 1,
wherein the interface stabilizer is a polyurethane polymer, which is formed by the reaction of a prepolymer (i) with a coreactant (ii), and optionally is terminated by an end-capping agent,
wherein the prepolymer (i) is formed by the reaction of a polyhydroxyl compound, polyisocyanates, and optionally low molecular weight diols which are optionally substituted with ionic groups or potential ionic groups.

3. The foaming composition according to claim 2,
wherein the polyhydroxyl compound is selected from the group consisting of polyester polyols;
the polyisocyanate is selected from the group consisting of aliphatic, cycloaliphatic and aromatic polyisocyanates,
the coreactant is selected from the group consisting of compounds containing functional groups, adapted to react with isocyanate groups in preference to the carboxyl group; and/or
the end-capping agent is selected from the group consisting of monoamines, alkanolamines, amide-amines, C₁-C₁₀ alcohols, and fatty alcohols.

4. The foaming composition according to claim 1, wherein the interface stabilizer is present in an amount ranging from 0.01 wt. % to 10 wt. %, relative to the total weight of the composition.

5. The foaming composition according to claim 1, wherein the at least one alkyl sulfosuccinate is selected from the group consisting of mono- or di-alkyl sulfosuccinates in which the alkyl radicals have 4 to 24 carbon atoms, and the counterion to the sulfonic acid group is selected from the group consisting of alkali metal cations and ammonium ions; and
the at least one alkyl sulfosuccinate is present in an amount ranging from about 0.5 wt. % to about 15 wt relative to the total weight of the composition.

6. The foaming composition according to claim 1, wherein the amphoteric surfactant is selected from the group consisting of optionally quaternized secondary or tertiary aliphatic amine derivatives; and
the amphoteric surfactant is present in an amount ranging from 0.5 wt. % to 20 wt. %, relative to the total weight of the composition.

7. The foaming composition according to claim 1, wherein the non-ionic surfactant is an alkyl poly glucoside having a C₆-C₃₄ alkyl group and a moiety derived from a reducing saccharide containing from 5 to 6 carbon atoms, and
the non-ionic surfactant is present in an amount ranging from 0 wt. % to 15 wt. %, relative to the total weight of the composition.

8. The composition according to claim 1, wherein the at least one oil is selected from the group consisting of oils of plant or animal origin, ester oils, ether oils, silicone oils, hydrocarbon oils, and mixtures thereof.

9. The composition according to claim 8, wherein the least one oil comprises at least one oil of plant or animal origin and/or at least one ester of saturated C₁₀-C₂₀ aliphatic monoacid(s) and of saturated C₂-C₁₀ aliphatic monoalcohol(s), and optionally at least one silicone oil and/or at least one hydrocarbon oil.

10. The composition according to claim 8 or 9, wherein the at least one oil is present in an amount of higher than 10 wt relative to the total weight of the composition, and
when the silicone oil(s) and/or hydrocarbon oil(s) are present, the amount of each of the silicone oil and hydrocarbon oil is equal to or lower than 10 wt. %, relative to the total weight of the composition.

11. The composition according to claim 1, wherein the weight ratio of the interface stabilizer to the at least one oil ranges from 1:500 to 1:1.

12. The composition according to claim 1, further comprising at least one aqueous phase.

13. A composition comprising, a surfactant system and at least one interface stabilizer capable of improving rheology of the composition and boosting and stabilizing the foams formed therein,
wherein the interface stabilizer is a polyurethane polymer, and the surfactant system consists of mono- or di-alkyl sulfosuccinate, (C₈-C₂₀) alkylamido (C₁-C₆) alkylbetaine, and alkyl poly glucoside;
the “high-oil product” means the oil is present in an amount of higher than 10 wt. %, relative to the total weight of the product.

14. A foaming composition for cleansing and/or removing makeup from keratin materials, comprising the composition according to claim 13 and at least one oil selected from the group consisting of oils of plant origin, ester oils, dimethicones, linear or branched hydrocarbons, and mixtures thereof.

15. A method to improve the rheology of an emulsified system and boosting and stabilizing the foams formed therein employing a polyurethane polymer, formed by the reaction of a prepolymer (i) with a coreactant (ii), and optionally is terminated by an end-capping agent (iii), wherein the prepolymer (i) is formed by the reaction of polyhydroxyl compounds, polyisocyanates, and optionally low molecular weight diols.
